(19) United States
(12) Patent Application Publication
(10) Pub. No.: US 2006/0010584 A1
(43) Pub. Date: Jan. 19, 2006

(54) Goggles for swimming or diving or surfing and a manufacturing method thereof

(76) Inventor: Ruisheng Liang, Guangzhou City (CN)

Correspondence Address: THORPE NORTH & WESTERN, LLP., 8180 SOUTH 700 EAST, SUITE 200, SANDY, UT, 84070, US

(21) Appl. No.: 10/521,840

(22) PCT Filed: Aug. 19, 2002
(86) PCT No.: PCT/CN02/00577

§ 371 Date: Aug. 18, 2005

(30) Foreign Application Priority Data
Jul. 25, 2002 (CN) 02134444.2

(51) Int. Cl. A61F 9/02

(52) U.S. Cl. 2/426

(57) ABSTRACT

The invention provides a pair of goggles for swimming or diving or surfing and a manufacturing method thereof. The goggles comprise of two frames, two lenses and strap. The lenses and the frames are moulded integrally by a high rigid PC and a silica gel material. The manufacturing method includes the following steps: To put the PC lenses inside the soft veil-shaped cavity; To inject a soft veil silica gel material into the cavity, when the injecting temperature is controlled below 250° C. and the injecting pressure is controlled at 50 T/cm². Since the goggle is a kind of once moulded product, the manufacturing processes are reduced, and the waterproof and gas-proof effect is effectively increased.

### TECHNOLOGICAL FIELD OF THE INVENTION

The invention relates to a kind of sport article, particularly, a kind of goggles for swimming, diving or surfing and its manufacturing method thereof.

### EXISTING TECHNOLOGY PRIOR TO THE INVENTION

The existing goggles for diving or surfing or swimming are customarily comprised of four parts, namely, external frame, soft veil, lenses, and compression framework. The soft veil is mostly made of PVC, TPR or silica gel, while the glass is mostly made of PC or toughened glass. The various complicated parts must be assembled with mature craftsmanship, and the finished products are not 100% waterproof or gas-proof. Before shipped out of the plant, the products need long time pressure test.

### PURPOSE OF THE INVENTION

The purpose of the invention is to overcome the above-mentioned disadvantages of the existing products and provide a kind of once moulded product, which can be one hundred percent waterproof and gas proof, which manufacturing process thereof can be simplified. Another purpose of the invention is to provide the manufacturing process thereof.

### TECHNOLOGICAL SCHEME OF THE INVENTION

The invention achieves its purpose in the following ways. The goggles comprise of two frames, two lenses and strap. The lenses and the frames are moulded integrally by a high rigid PC and a silica gel material.

The manufacturing method includes the following steps: To put the PC lenses inside the soft veil-shaped cavity; To inject a soft veil silica gel material (a kind of high rigid joint molding material) into the cavity, when the injecting temperature is controlled between 200˜250° C. and the injecting pressure is controlled at 40˜50 T/cm².

### ADVANTAGES OF THE INVENTION

(1) Fastness: the principal part of the glass is molded at one time through injecting, heating, pressing and opening, so the glass and the soft veil are very firmly united, and they will never fall apart when you use it or impact it.
(2) Tightness: since the glass and the silica gel are united at one time through injection, the product is totally waterproof and gas proof when swimming, diving and surfing.
(3) Simpleness: the principal part of the glass is once moulded integrally, which manufacturing processes are reduced and the long time air and water leak test is eliminated.

### FIGURE DESCRIPTION

The basic operating mode of the invention will be further described with respect to the attached figure.

FIG. 1 is the schematic drawing of the structure of the invention.

### EMBODIED EXAMPLE

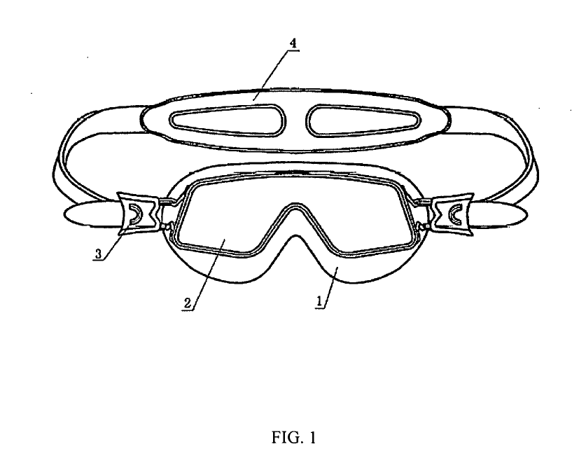

As FIG. 1 shows, the product is composed of Frame 1, lenses 2, Adjustment Button 3 and strap 4. The principal part of the invention is comprised of Frame 1 and lenses 2. Lenses 2 and Frame 1 are molded integrally with high rigid PC and silica gel.

The manufacturing process of the invention includes the following steps: To place the PC lenses into the cavity of the soft veil-shaped mold, and inject silica gel (high-rigid joint molding material), which is the raw material of the soft veil. The injection temperature shall be 250° C. or below, and the gel injection pressure shall be 50 T/cm². Through the continuous process flow of injecting, heating, pressing and opening, the product is firmly established and not easy to twist.

1. A pair of goggles for swimming, diving or surfing, comprising of frames(1), lenses (2) and strap, the lenses (2) and the frames(1) being moulded integrally by a high rigid PC and a silica gel material.

2. A method to manufacture the goggles in accordance with claim 1, includes the following steps: to put the PC lenses inside the soft veil-shaped cavity; to inject a soft veil silica gel material (a kind of high rigid joint molding material) into the cavity, when the injecting temperature is controlled bellow 200˜250° C. and the injecting pressure is controlled at 40˜50 T/cm².

3. A method in accordance with claim 2, the injecting temperature shall be controlled bellow 250° C., and the injecting pressure shall be controlled at 50 T/cm²
